(19) United States
(12) Patent Application Publication
(10) Pub. No.: US 2006/0264838 A1
(43) Pub. Date: Nov. 23, 2006

(54) Device for expelling a liquid or pasty substance

(76) Inventors: Jean-Claude Volckmann, Bonne (FR); Yannick Rosset, Celigny (CH)

Correspondence Address: Ronald R Santucci;Frommer Lawrence & Haug, 745 Fifth Avenue, New York, NY, 10151, US

(21) Appl. No.: 10/564,247

(22) PCT Filed: Jul. 15, 2004
(86) PCT No.: PCT/IB04/02297

§ 371 Date: Apr. 24, 2006

(30) Foreign Application Priority Data
Jul. 16, 2003 (FR) 0308686

(51) Int. Cl. A61M 5/00

(52) U.S. Cl. 604/209; 433/89

(57) ABSTRACT

The device (1) for ejecting a liquid or pasty product, comprises a body (2), a part intended to contain the product and equipped with an orifice for ejecting the product, a drive cylinder (3) equipped with teeth (10), moving in a bore (23) passing through the body and causing the volume of the part intended to contain the product to vary and a mechanism for displacing the drive cylinder attached to the body, and comprises an articulated lever (4) acting on the teeth of the drive cylinder through an articulated pawl (16) articulated to the lever (4) and returned to a position of contact with the drive cylinder and a nonreturn pawl (18) returned to a position of contact with the drive cylinder. It is one wherein the lever (4) is connected to the body by means of a joint (14, 15) that can be dislocated.

### BACKGROUND OF THE INVENTION

The invention relates to a device for ejecting a liquid or pasty product, comprising a body, a part intended to contain the product and equipped with an orifice for ejecting the product, a drive cylinder equipped with teeth, moving in a bore passing through the body and causing the volume of the part intended to contain the product to vary and a mechanism for displacing the drive cylinder attached to the body, comprising an articulated lever acting on the teeth of the drive cylinder through an articulated pawl articulated to the lever and returned to a position of contact with the drive cylinder and a nonreturn pawl returned to a position of contact with the drive cylinder.

### DESCRIPTION OF THE PRIOR ART

This type of device is known from patent U.S. Pat. No. 4,444,560 or from patent application FR 2 535 206 which describes a dental syringe for intra-ligamental injection. This syringe allows a very fine and flexible needle to inject product into the ligaments between the jawbone and the tooth. It consists mainly of an elongate body on which is mounted a mechanism controlling injection through the displacement of a drive cylinder, a container holder in which a container full of liquid for injection is housed, and a nozzle comprising the injection needle. In order to solve the problems connected with the difficulty of accessing the areas to which the injections are to be administered, the body of the syringe has an injection head that is at an angle to the axis of the body of the syringe. The needle, which is removable, is fitted onto the body before the injections are administered, then withdrawn afterwards. The mechanism controlling injection is mainly made up of a lever articulated to the syringe body and acting on a drive cylinder through an articulated pawl articulated to the lever and returned to a position of contact with the teeth of a rack produced on the drive cylinder. The cylinder is guided in translational movement in a bore made in the body of the syringe. It also has a longitudinal groove collaborating with a screw screwed in radially with respect to the bore and opening into the latter to prevent the cylinder from turning. The mechanism additionally comprises a nonreturn pawl preventing the drive cylinder from retreating when action on the lever ends. This nonreturn pawl is returned to a position of contact with the teeth of the rack and may be moved away from this position by action on a button in order to cancel the injection pressure and/or in order to change the container of product for injection against which the drive cylinder presses.

A device such as this has disadvantages: on the one hand, it is complicated to produce. On the other hand, it has a great many parts and complicated shapes, particularly angles and corners in the material. These angles and corners form regions which are very difficult to access and as a result very difficult to clean and therefore difficult to sterilize.

### SUMMARY OF THE INVENTION

The object of the invention is to propose a device for ejecting a liquid or pasty product that alleviates the mentioned disadvantages and improves the devices known from the prior art. In particular, the invention proposes to produce a device that is of simple construction, easy to disassemble and to clean.

The ejection device according to the invention is one wherein the lever is connected to the body by means of a joint that can be dislocated. This characteristic makes it possible to ensure ease of access for cleaning the various parts of the device.

Various embodiments of the device are defined by dependent claims 2 to 10.

### DESCRIPTION OF THE DRAWINGS

The attached drawing depicts, by way of examples, two embodiments of a product-ejection device according to the invention.

FIG. 1 is a view of one embodiment of a liquid-ejection device according to the invention.

FIG. 2 is a view in section of this embodiment.

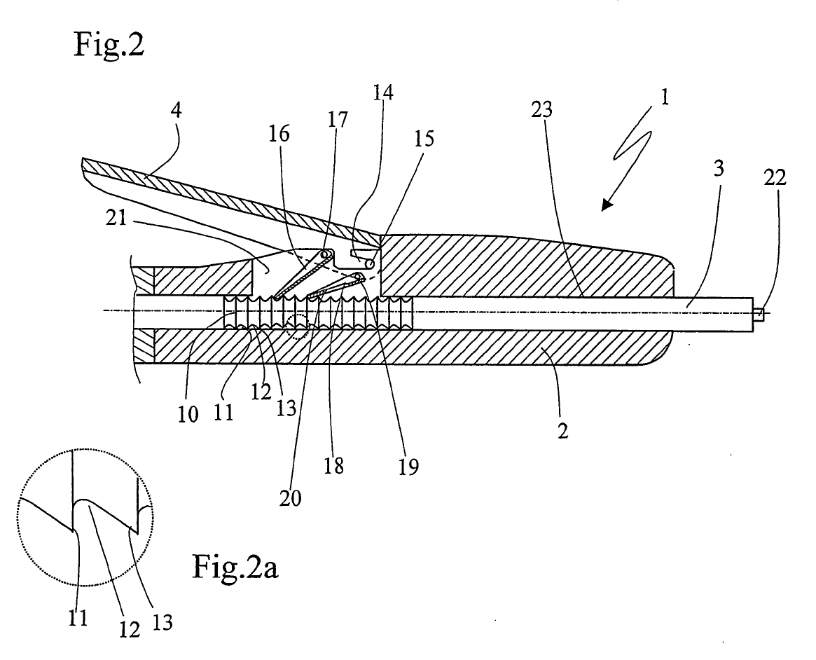

FIG. 3 is a view of a first embodiment variant of the drive cylinder.

FIG. 4 is a view of a second embodiment variant of the drive cylinder.

FIGS. 5 and 6 are views in section of the second embodiment variant of the drive cylinder.

FIG. 7 is a view in section of a second embodiment of a liquid-ejection device according to the invention.

### DESCRIPTION OF THE PREFERRED EMBODIMENTS

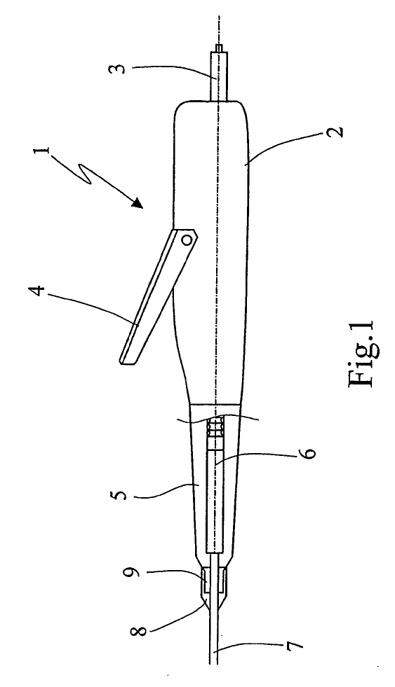

The device 1 for ejecting a product, depicted in FIG. 1, mainly comprises a body 2 on which a container holder 5 is fixed.

The container holder 5 has a cylindrical cavity 6 intended to accept either a container full of product for ejecting, or the product for ejecting itself directly. The end of the container holder has a channel in which there is removably attached a tube 7 connected, for example, to a tapped ring 8 or to a ring exhibiting some other fastening system. Fastening is by screw-fastening the tapped ring onto a threaded nozzle 9 produced on the container holder around the channel.

The body 2 comprises a mechanism for ejecting and metering the product. This mechanism has a lever 4 articulated to the body about a pivot pin 15. This lever is connected to the body by a joint produced by elastic deformation of its pivot pin 15 as it fits into slots 14 made in the body 2. This joint makes for ease of disassembly of the lever preferably requiring no disassembly tools and therefore allowing access for cleaning the lever and those regions of the body that lie under the lever. It also allows the user to mount on the body a lever the size of which is tailored to his or her build. This makes it possible to improve the precision with which the device can be used. This lever for example consists of a U-section the two lateral flanges of which are pierced to allow the pivot pin 15 to pass. The lever allows operation on a drive cylinder 3 made, for example, of stainless steel and having teeth 10 which can have symmetry of revolution and the profile of which consists of two straight segments 11 and 13 connected via a radius 12. Such a toothed profile makes it possible to avoid creating areas to which access is tricky and which are difficult to clean. In particular, the tooth trough radius is large enough to minimize the risk of dirt becoming caught in the troughs of the teeth and to make access with cleaning equipment easier. The magnitude of the radius is, for example, of the order of magnitude of the depth of the teeth. The two segments could, with respect to the axis of the cylinder, make angles which also facilitate access with cleaning equipment. The cylinder 3 is guided in translation in the body 2 by a bore 23. Its end located in the cylindrical cavity is able to push back either a wall of the container full of product for ejecting or the product for ejecting itself directly. The bore 23 opens on either side of the body 2. It is thus perfectly accessible and exhibits no regions that are difficult to clean.

The ejection mechanism comprises a removable pawl 16, articulated to the lever 4 about a pivot pin 17 and returned by a spring, not depicted, to a position of contact with the teeth of the cylinder 3. This return force ensures constant contact between the pawl 16 and the cylinder, on the one hand, and returns the lever 4 to a position away from the body 2 from which position it is able to exert an action capable of ejecting some of the product contained in the container holder.

The ejection mechanism also comprises a nonreturn pawl 18 articulated to the body 2 about a pivot pin 19 and returned by a spring, not depicted, to a position of contact with the teeth of the cylinder 3. This nonreturn pawl prevents the drive cylinder from retreating when the user releases the pressure exerted on the lever 4 so that the lever returns to its position away from the body. This nonreturn pawl may in addition exhibit an opening 20 collaborating with a projection 22 provided at one of the ends of the cylinder to prevent the latter from being inserted into the bore the wrong way round. The ejection mechanism could also exhibit a structure such that the lever acts directly on the teeth of the drive cylinder.

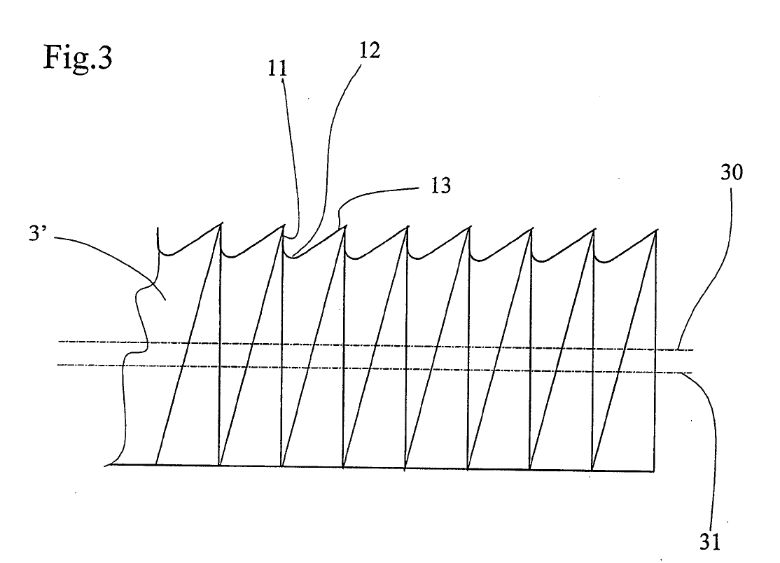

In a first embodiment variant of the drive cylinder 3′, depicted in FIG. 3, the teeth have symmetry of revolution but the distance from the axis of revolution 31 of the profile to the profile is greater than the radius of the cylinder. In this way, the drive cylinder has a sector with no teeth along its entire length. This allows the pressure in the container holder to be canceled quickly by turning the drive cylinder.

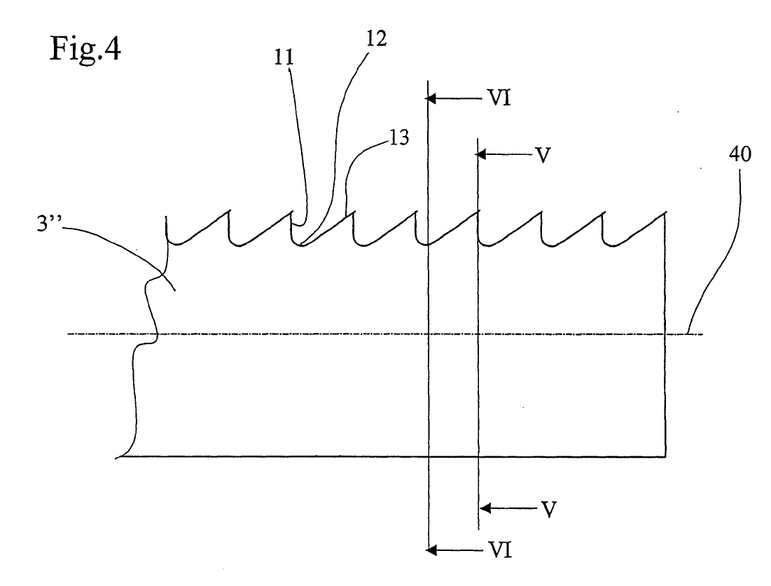

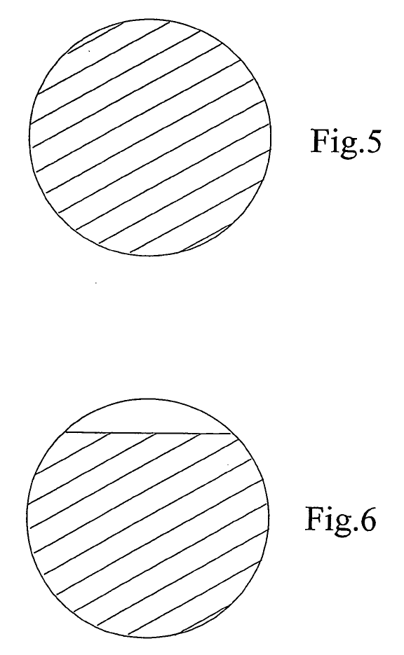

In a second embodiment variant of the drive cylinder 3″, depicted in FIG. 4, the teeth constitute a regulated surface the generatrices of which are, in the region of the teeth, more or less orthoradial with respect to the drive cylinder. The drive cylinder thus also has a sector free of teeth over its entire length and allows the pressure in the container holder to be canceled quickly by turning the drive cylinder. The cross section of the cylinder is depicted at the crest of a tooth in FIG. 5 and at the trough of a tooth in FIG. 6.

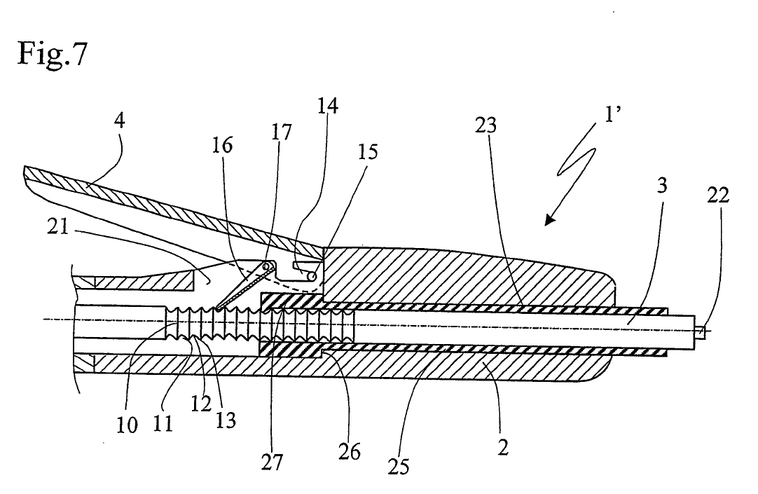

A second embodiment of the device according to the invention is depicted in FIG. 7. This embodiment differs from the previous one in that the bore 23 has a step 26 at the slot 21 made in the body 2. The bore accepts a liner 25 made for example of polytetrafluoroethylene or some other plastic such as a polyetheretherketone (PEEK). This liner comprises, on its outside diameter, a step collaborating with the step 26 of the bore to halt its translational movement. This liner is a sliding fit in the body. At the slot 21 it comprises two axial slits opening at its end to produce a stud 27 the end of which comes into contact with the teeth 10 of the drive cylinder. The stud is returned to this position by the forces of elastic deformation of the material of which it is made. Such an embodiment makes it possible to simplify the construction of the device and thus make it easier to clean. With such a structure, in order to cancel the product-ejection pressure, there is no longer any need to turn the drive cylinder relative to the body. Indeed, all that is required is for the drive cylinder to be turned with respect to the liner, it being possible in particular for this movement to be obtained by turning the liner with respect to the body, the drive cylinder being fixed relative to the body. Turning the liner with respect to the body is easier than turning the drive cylinder. This is because the forces applied to the end of the drive cylinder may be such that these, combined with the coefficient of friction between the drive cylinder and the container, make this rotation impossible. The nonreturn pawl may consist of a deformable part of the body just as it may consist of a deformable part of the liner.

In embodiment variants, the liner may exhibit studs for releasing the drive cylinder on its outside diameter and be capable of rotational or translational movement relative to the body. These movements allow the release studs to act on the various pawls in order to bring them into a position that releases the drive cylinder. The liner may also exhibit means allowing it to make a connection between the body and the part intended to contain the product. For example, it may at one of its ends exhibit studs to allow it to be assembled with the part intended to contain the product by a bayonet-type connection and at the other of its ends may exhibit a step holding the body against the part intended to contain the product.

The materials of which the various parts of the device are made may be chosen from products compatible with sterilization methods.

Such a device may be used in the field of medicine for injecting products such as anesthetics into hard tissue or for depositing adhesives, resins or amalgams. It may also be used in the paramedical field for depositing determined amounts of collagen. It may furthermore be used in the field of micromechanics and jewelry to make bonded connections or microwelds or alternatively for depositing products.

1. A device (1) for ejecting a liquid or pasty product, comprising a body (2), a part intended to contain the product and equipped with an orifice for ejecting the product, a drive cylinder (3; 3′; 3″) equipped with teeth (10), moving in a bore (23) passing through the body and causing the volume of the part intended to contain the product to vary and a mechanism for displacing the drive cylinder attached to the body, comprising an articulated lever (4) acting on the teeth of the drive cylinder through an articulated pawl (16) articulated to the lever (4) and returned to a position of contact with the drive cylinder and a nonreturn pawl (18) returned to a position of contact with the drive cylinder, wherein the lever (4) is connected to the body by means of a joint (14, 15) that can be dislocated.

2. The ejection device (1) as claimed in claim 1, wherein the drive cylinder (3; 3′; 3″) has one end (22) shaped to engage in a slot (20) formed on the nonreturn pawl (18) when the latter is engaged the wrong way round in the body (2).

3. The ejection device (1) as claimed in claim 1, wherein the drive cylinder (3′; 3″) has a sector with no teeth along its entire length and is able to rotate in the bore (23).

4. The ejection device (1) as claimed in claim 1, wherein the profile of the teeth (10) between two consecutive crests comprises two straight segments (11, 13) connected by a radius (12).

5. The ejection device (1) as claimed in claim 1, and which comprises, in the body (2), a liner (25) able to rotate with respect to the body.

6. The ejection device (1) as claimed in claim 5, wherein the liner comprises an elastically deformable part (27) constituting the nonreturn pawl.

7. The ejection device (1) as claimed in claim 5, wherein the liner comprises at least one stud designed to act on at least one pawl and to release the drive cylinder as the liner turns.

8. The ejection device (1) as claimed in claim 1, and which comprises, in the body (2), a liner (25) capable of translational movement with respect to the body (2) and wherein the liner comprises at least one stud designed to act on at least one pawl and release the drive cylinder as the liner effects a translational movement.

9. The ejection device (1) as claimed in claim 5, wherein the liner comprises a means of connection to the part intended to contain the product.

10. The ejection device (1) as claimed in claim 1, wherein the body (2) and/or the lever (4) are made of a material that can be molded.
